(19) United States
(12) Patent Application Publication
(10) Pub. No.: US 2018/0147304 A1
(43) Pub. Date: May 31, 2018

(54) Risk Stratification

(71) Applicant: GE HEALTHCARE LIMITED, Buckinghamshire (GB)

(72) Inventors: Jean-Claude Provost, Buckinghamshire (GB); Paul Alexander Jones, Buckinghamshire (GB); Etienne Montagut, Buckinghamshire (GB); Jay Chahal, Buckinghamshire (GB); Meridith Keating Johnson, Buckinghamshire (GB)

(73) Assignee: GE HEALTHCARE LIMITED, Buckinghamshire (GB)

(21) Appl. No.: 15/576,267

(22) PCT Filed: May 23, 2016
(86) PCT No.: PCT/EP2016/061608

§ 371 Date: Nov. 21, 2017

(30) Foreign Application Priority Data
May 22, 2015 (GB) 1508844.6
Jun. 4, 2015 (GB) 1509681.1

(51) Int. Cl. A61K 51/04; A61P 9/00

(52) U.S. Cl. CPC A61K 51/0406; A61P 9/00

(57) ABSTRACT

The present invention relates to a method of in vivo imaging with meta-[¹²³I]iodobenzylguanidine ([¹²³I]mIBG) and more particularly wherein said method is used to stratify a defined subset of subjects with heart failure into particular treatment regimens.

### TECHNICAL FIELD OF THE INVENTION

The present invention relates to the field of medical imaging of human subjects. In particular the invention relates to cardiac neurotransmission imaging in said subjects and provides a novel imaging method that permits improved management of said subjects.

### DESCRIPTION OF RELATED ART

Approximately 5.1 million people in the United States of America (USA) have clinical manifestation of heart failure (HF) and the prevalence continues to increase (Yancy et al. 2013 JACC; 62(16)). Similarly, the European Society of Cardiology estimates that 15 million Europeans suffer from HF with this number expected to rise to 30 million by 2020. Chronic HF is associated with high morbidity and mortality; the absolute mortality rate for HF remains at approximately 50% within 5 years of diagnosis.

In addition to medical therapies, device therapies such as implantable cardioverter defibrillators (ICD) are available and have proven effectiveness, especially for improving HF symptoms, preventing sudden cardiac death via the reduction of arrhythmic events. However, implant and care of ICD is expensive. In 2006, costs per implant per patient in the USA were estimated at 28,500-55,200 and annual follow up S4,800-17,000 (Groeneveld et al. 2006 Am J Cardiol; 98(10): 1409-1415). A more recent study in the Netherlands found a life time counts for an ICD to range from €60,800-64,200 (Thijssen et al. 2014 Pacing Clin Electrophysiol; 37 (1): 25-34).

Based on the available evidence, some efforts have been made to improve risk-stratification of patients prior to ICD implantation. Current USA and European Union (EU) guidelines (see e.g. Yancy et al. 2013 JACC; 62(16): e147-239) recommend implantation of an ICD in all patients with LVEF≤35% and New York Heart Association (NYHA) functional class II or III to reduce the risk of sudden cardiac death.

Even so, during the first year of implantation, as few as 5% of patients who have a device implanted will actually experience an appropriate shock or pace termination of a fatal arrhythmia (Moss et al. 2012 New Engl J Med; 367(24): 2275-2283). Thus, the majority of patients who do have a device implanted do not actually benefit from the device. These patients are also not immune to complications of device implantation and on-going care, including mechanical and infectious complications of the procedure itself, inappropriate device activation, device failure, and need for device battery replacement every several years (Kirkfeldt et al. 2014 European Heart Journal; 35: 1186-1194; Buber et al. 2014 Europace; 16: 227-234). Despite optimal therapy and device care, approximately 2% of HF patients with devices will die suddenly each year, and 3% will die from other cardiac causes, usually HF or myocardial infarction. In addition, inappropriate shocks administered by the ICD appear to be associated with a higher incidence of all cause and cardiovascular death (Moss et al. supra). Al-Khatib et al. (2014 International Journal of Cardiology; 172(1): 253-254) suggested that devices for primary prevention of sudden cardiac death should only be implanted in patients with LVEF<30% suggesting that the population of patients with LVEF between 30% and 35% may not benefit from ICD implantation.

Optimizing risk stratification and appropriately triaging individual patients to these invasive options to improve clinical outcomes remains a clinical challenge (Yancy et al. supra; Goldberger et al. 2011 Circulation; 123: 2423-2430).

The prognostic value of meta-[¹²³I]iodobenzylguanidine ([¹²³I]mIBG) has been investigated in a number of studies. In 2008 Verberne et al. reviewed the data from eighteen meta-[¹²³I]iodobenzylguanidine ([¹²³I]mIBG) studies (2008 Eur Heart J; 29: 1147-1159) to determine its prognostic significance. The review concluded that patients with heart failure and decreased late H/M or increased myocardial [¹²³I]mIBG washout have a worse prognosis compared with those with normal semi-quantitative myocardial mIBG parameters. However, these conclusions apply to a wide variety of conditions without any specific guidance about patient population, how the test should be performed or how results guide any medical treatment decision. The ADMIRE-HF study examined the predictive value of the [¹²³I]mIBG formulation AdreView™ (Jacobson et al. 2010 J Am Col Cardiol; 55(20): 2212-2221). This study evaluated the heart/mediastinal uptake ratio (H/M) at 4 hours after [¹²³I]mIBG injection as a risk factor for HF progression, arrhythmic events (sustained ventricular tachycardia, cardiac arrest, or appropriate ICD discharge), or cardiac death. An H/M ratio of <1.6 was independently associated with cardiac events after adjustment for LVEF, b-type natriuretic peptide (BNP), and NYHA functional class. Whether [¹²³I]mIBG is useful in selection of patients for ICD implantation has yet to be established.

### SUMMARY OF THE INVENTION

In a first aspect, the present invention relates to a method comprising:
(i) obtaining an in vivo image with meta-[¹²³I]iodobenzylguanidine ([¹²³I]mIBG) of a human subject wherein said subject has a left ventricular ejection fraction (LVEF) of 30-35% and New York Heart Association (NYHA) symptoms of class II or above;
(ii) measuring [¹²³I]mIBG uptake in said subject wherein said measuring comprises defining in said in vivo image a heart region of interest (ROI) and a mediastinum ROI;
(iii) calculating the ratio of [¹²³I]mIBG uptake between said heart ROI and said mediastinum ROI (H/M ratio);
(iv) making a clinical decision based on whether or not H/M is less than 1.6 wherein said clinical decision is to implant a cardioverter-defibrillator (ICD) only where said H/M ratio is less than 1.6.

In a second aspect, the present invention relates to use of [¹²³I]mIBG in a method for guiding ICD therapy in a group of patients having LVEF of 30-35% and NYHA symptoms of class II or above wherein said method is the method of the first embodiment of the invention.

In a third aspect, the present invention relates to a method comprising:
(i) obtaining an in vivo image with meta-[¹²³I]iodobenzylguanidine ([¹²³I]mIBG) of a human subject wherein said subject has a left ventricular ejection fraction (LVEF) of 30-35% and New York Heart Association (NYHA) symptoms of class II or above;
(ii) measuring [¹²³I]mIBG uptake in said subject wherein said measuring comprises defining in said in vivo image a heart region of interest (ROI) and a mediastinum ROI;
(iii) calculating the ratio of [¹²³I]mIBG uptake between said heart ROI and said mediastinum ROI (H/M ratio);
(iv) making a clinical decision based on whether or not H/M is less than 1.6 wherein said clinical decision is selection of subjects for a clinical trial.

In a fourth aspect, the present invention provides a composition comprising [¹²³I]mIBG for use in the method of the first embodiment or the use of the second embodiment. The present invention provides additional guidance to clinicians seeking to direct ICD implantation to those patients who will experience the most benefit, thereby saving time, effort and cost as well as avoiding the potentially fatal risks associated with ICD implantation in patients who are unlikely to experience any benefit.

### BRIEF DESCRIPTION OF THE FIGURES

FIG. 1 is a Kaplan Meier curve for all-cause mortality at 5 years follow up comparing patients with AdreView™ H/M ratio <1.6 and ≥1.6 in the LVEF strata of 30-35%.

FIG. 2 is a Kaplan Meier curve for cardiac mortality at 5 years follow up comparing patients with AdreView™ H/M ratio <1.6 and ≥1.6 in the LVEF strata of 30-35%.

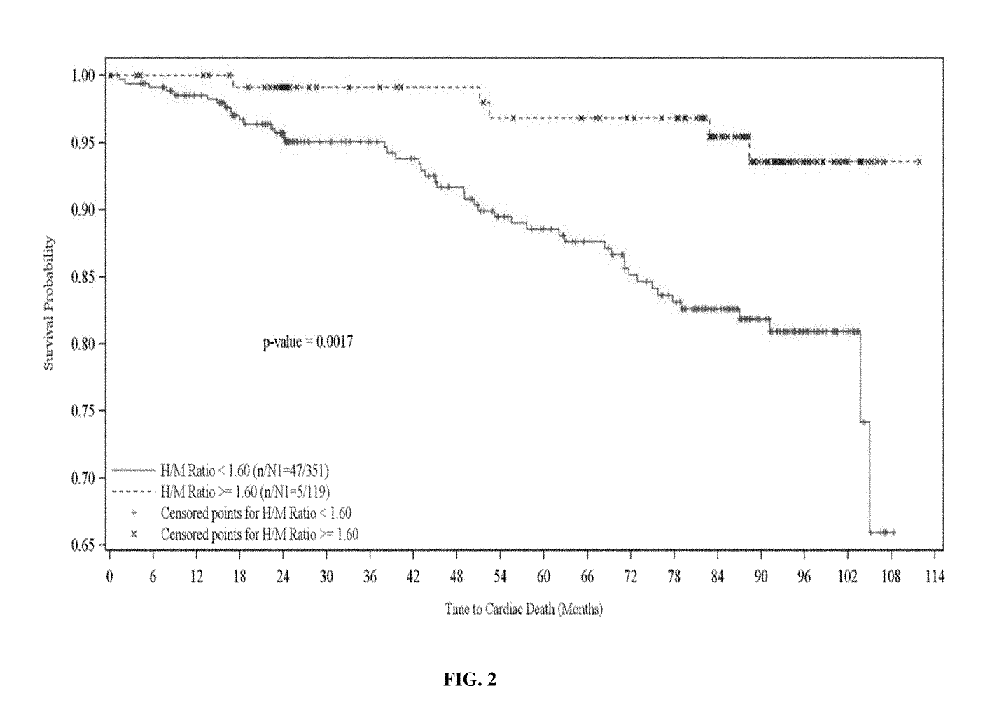

FIG. 3 is a Kaplan Meier curve for time to first arrhythmic event based on the on-site LVEF categorization (>=30%-35%) and AdreView™ uptake H/M ratio (<1.60 vs. >=1.60).

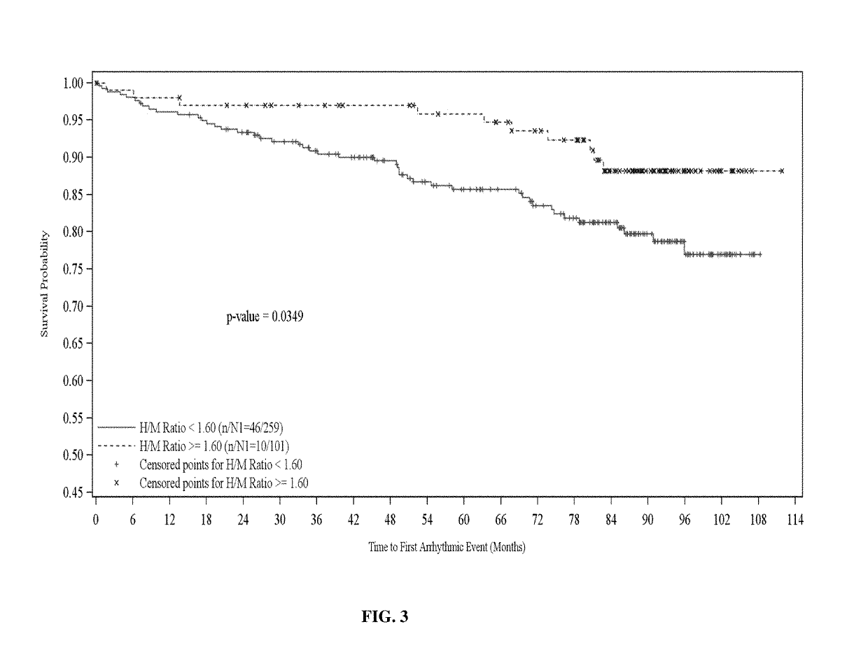

### DETAILED DESCRIPTION OF THE PREFERRED EMBODIMENTS

To more clearly and concisely describe and point out the subject matter of the claimed invention, definitions and particular embodiments are provided hereinbelow for the terms used throughout the present specification and claims. Any exemplification of specific terms herein should be considered as a non-limiting example.

“Meta-iodobenzylguanidine (mIBG)” is a synthetic guanethidine analogue similar to norepinephrine (NE). The ¹²³I-labelled version ([¹²³I]mIBG) has the following structure:

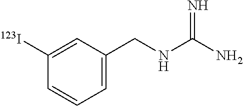 [SMILES: N=C(N)NCc1cccc([123I])c1]

Imaging with [¹²³I]mIBG is an established test to evaluate innervation of the cardiac sympathetic nervous system. As a structural and functional analogue of NE, [¹²³I]mIBG is subject to the same uptake and accumulation pathways. [¹²³I]mIBG is primarily taken up from the extracellular environment into the pre-synaptic sympathetic nerve terminals by the NE transporter (NET). Similar to NE, [¹²³I]mIBG is subsequently stored in pre-synaptic vesicles, but in contrast to NE it does not undergo metabolism. This accumulation of [¹²³I]mIBG enables the in vivo imaging of sympathetic innervation of adrenergic nerves such as those of the heart. [¹²³I]mIBG is commercially-available from GE Healthcare as the radiopharmaceutical formulation AdreView™.

“Left ventricular ejection fraction (LVEF)” is the fraction of blood pumped out of the left heart with each heartbeat. A normal value for LVEF is over 50% but in patients with heart failure, the LVEF is typically below 40% and in many cases below 30%.

“New York Heart Association (NYHA) class” is a well-established measure of the extent of heart failure in patients. Classes I-IV can be understood as follows:

| NYHA | |
|---|---|
| Class | Symptoms |
| I | Cardiac disease, but no symptoms and no limitation in ordinary |
| | physical activity, e.g. no shortness of breath when walking, |
| | climbing stairs etc. |
| II | Mild symptoms (mild shortness of breath and/or angina) and |
| | slight limitation during ordinary activity. |
| III | Marked limitation in activity due to symptoms, even during |
| | less-than-ordinary activity, e.g. walking short distances |
| | (20-100 m). Comfortable only at rest. |
| IV | Severe limitations. Experiences symptoms even while at rest. |
| | Mostly bedbound patients. |

In one embodiment, said NYHA class is present while the subject is receiving guideline-directed medical therapy (GDMT). In one embodiment said NYHA class is III or above. In one embodiment said NYHA class is IV.

The term “[¹²³I]mIBG uptake” refers to the amount of [¹²³I]mIBG retained in tissues of the subject at a defined time following its administration to said subject. [¹²³I]mIBG uptake can be understood to be the actual uptake in said subject as well as uptake as illustrated in the in vivo image obtained in step (i) of the method of the invention.

The term “defining” with respect to defining a region of interest (ROI) refers to the process comprising drawing an area on the in vivo image corresponding to that ROI. In one embodiment the in vivo image is a digital image represented on a computer screen and drawing is achieved by means of a suitable software package.

The step of “making a clinical decision” based on whether or not H/M is less than 1.6 can encompass a range of clinical decisions, e.g. in clinical trials for stratification of subjects or in clinical treatment of patients. In one embodiment, said clinical decision is selection of subjects for a clinical trial. The statistics in clinical trials are dependent on the patient cohort in the treated and untreated arms having cardiac events (e.g. potentially fatal arrest) and by using the method of the present invention high risk patients can be identified and placed into the trial, which increases the event rate and reduces the number of subjects required to demonstrate efficacy in the trial. In one embodiment, said is clinical decision is to implant a cardioverter-defibrillator (ICD) where said H/M ratio is less than 1.6. In another embodiment said clinical decision is not to implant an ICD where said H/M ratio is greater than or equal to 1.6. The term “implantable cardioverter-defibrillator (ICD)” refers to a medical device used in the treatment of heart failure that comprises a generator placed under the skin of the left upper chest and wires connected to the generator that are passed through a vein to the right chambers of the heart. The device is configured to perform cardioversion, defibrillation and pacing of the heart. ICDs can also have an anti-tachycardia pacing function which refers to the use of pacing stimulation techniques for termination of tachyarrhythmias like ventricular tachycardia (VT). It is a painless way to terminate VT rather than using an electric shock. Sub-cutaneous ICDs (S-ICDs) are also now available, i.e. for which leads and wires are not inserted into the veins but rather are sub-cutaneous. These are easier to implant and less subject to complications. However by design these cannot have the antipacing function as leads/wires are sub-cutaneous.

In one embodiment of the method of the invention step (i) comprises intravenous injection of a radiopharmaceutical composition comprising [¹²³I]mIBG. The term “intravenous injection” refers to administration of a substance directly into the vein of a subject, typically using a hypodermic needle.

A “radiopharmaceutical composition comprising [¹²³I]mIBG” is a composition comprising [¹²³I]mIBG in a form suitable for human administration. In one embodiment said radiopharmaceutical composition comprises a biocompatible carrier. By the term “biocompatible carrier” is meant a fluid, especially a liquid, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer. Hence the aqueous suspension suitably excludes water-immiscible organic solvents.

In one embodiment of the method of the present invention step (i) comprises planar scintigraphy. The term “planar scintigraphy” images the distribution of an injected gamma-emitting isotope bound to a biologically active molecule (e.g. [¹²³I]mIBG) in a single two-dimensional image, analogous to a planar X-ray scan. The term “anterior planar scintigraphic image” will be well understood by those of skill in the art of in vivo imaging as an image taken from the front of the subject.

In one embodiment of the method of the present invention step (i) comprises obtaining early and late anterior planar scintigraphic images. The terms “early” and “late” in the context of anterior planar scintigraphic images of the present invention refer to those images taken following administration of [¹²³I]mIBG respectively before and after wash-out of [¹²³I]mIBG. The term “wash-out” refers to the elimination from organs and tissues of the subject of [¹²³I]mIBG that has not specifically been taken up by sympathetic neurons. In one embodiment said early anterior planar scintigraphic image is obtained at 15 minutes following injection of said radiopharmaceutical composition comprising [¹²³I]mIBG. In one embodiment said late planar scintigraphic image is obtained at 4 hours following injection of said radiopharmaceutical composition comprising [¹²³I]mIBG.

In one embodiment of the method of the invention wherein said heart can be visualised in said in vivo image, said heart ROI is defined by the epicardial border of the heart. The “epicardial border of the heart” refers to the innermost layer of the pericardium, in direct contact with the heart and thereby defining the outer border of the heart.

In one embodiment of the method of the invention wherein said heart cannot be visualised in said in vivo image, said heart ROI is defined by the presumed location of the heart. A person of skill in the art of cardiac in vivo imaging will be able to find in a straightforward manner the “presumed location of the heart” in those cases where the heart cannot be visualised in said in vivo image.

The “mediastinum” is the central compartment of the thoracic cavity surrounded by loose connective tissue. In one embodiment said mediastinum ROI is defined within the superior mediastinum. The “superior mediastinum” refers to that area of the mediastinum below the lung apices. In one embodiment, the mediastinum ROI is a defined section of the in vivo image within said superior mediastinum equidistant from the medial aspects of the right and left lung.

Defined methods of calculating the H/M ratio are known in the art. For example, the prescribing information for AdreView™ sets out in detail the steps that should be carried out using an in vivo image obtained by planar scintigraphy using AdreView™ (GE Healthcare AdreView™ Iobenguane I 123 Injection prescribing information March 2013).

In one embodiment of the method of the invention a mean count density is obtained for each of said heart ROI and said mediastinum ROI. The term “mean count density” refers to the mean counts per pixel of a particular ROI.

In one embodiment of the method of the invention said [¹²³I]mIBG imaging comprises single-photon emission tomography (SPECT) imaging “SPECT imaging” can be defined as a method of imagine that produces a series of contiguous two-dimensional images of the distribution of the radiotracer using the same agents as planar scintigraphy as defined above.

In an extension of ADMIRE-HF study (™ (Jacobson et al. 2010 J Am Col Cardiol; 55(20): 2212-2221)), Example 1 below describes a retrospective collection of survival data from heart failure (HF) patients with evaluable images recruited in ADMIRE HF, alive at last follow-up contact. If alive, investigators assessed changes in cardiac physiology (transplant or device insertion) and/or occurrence of arrhythmic events (resuscitated cardiac death and/or implantable defibrillator discharge). Cox proportional hazards model and Kaplan Meier curves were used. Subjects were dichotomized to <1.60 or ≥1.60 heart to mediastinum ratio (H/M) obtained in planar images at 3 h and 50 min A total of 964 patients were analyzed; 656 patients contributed new follow-up data (mean=57.87 months, median=62.7 months).

Subjects with H/M≥1.6 had significantly lower risk of cardiac death than those with H/M<1.6. Relative hazard ratio (HR) (time t) for 1-unit increase in the numerical H/M was 0.0732 (p<0.0001). Higher ratios significantly lowered the risk of a first arrhythmic event; HR for 1-unit increase in numerical H/M, 0.2968 (p=0.0046); HR, 0.4499; p=0.0012. Risk of death or potentially life-saving intervention was lower for subjects with H/M≥1.6; relative HR for 1-unit increase in numerical H/M of 0.2032 (p<0.0001); HR, 0.4585 (p<0.0001). For subjects with LVEF 30% to 35% (on-site measurement), all-cause mortality was lower for subjects with H/M≥1.6 (19.8%; 95% CI 12.5%-28.9%) vs H/M<1.6 (43.5%; 95% CI, 37.7%-49.5%). Cardiac mortality was higher for subjects with LVEF 5%-<30% and H/M<1.16 vs H/M≥1.6:81 (26.1%) vs 4 (6.6%); and for LVEF 30%-35%, 47 (16.5%) vs 5 (5%). Survival probability at 60 months from cardiac death was 84.2% for H/M<1.6 vs 96.4% H/M≥1.6; after first arrhythmic event, 81.8% vs 94.2%, and survival and time to death at 60 months or potentially life-saving intervention was 56.1%, vs 78.2%, respectively. Having H/M≥1.6 significantly lowered HR of cardiac death or fatal arrhythmia aborted by resuscitation or ICD discharge (HR, 0.4218; p=0.0002). The results suggest that HF patients with H/M<1.6 are at higher risk of death (all-cause and cardiac) and arrhythmic events for up to 5 years. The H/M may further discriminate patients at high risk of mortality independently of their LVEF. The present invention therefore can identify within HF patients having a LVEF between 30% and 35% those at low risk for cardiac death and therefore not requiring an ICD implantation. Advantages from a patient management and a health economic perspective are provided as only those requiring an ICD need to undergo what is a relatively invasive and expensive procedure.

The definitions and embodiments as defined for the first aspect of the invention as defined herein are equally applicable to the second aspect of the invention.

### BRIEF DESCRIPTION OF THE EXAMPLES

Example 1 describes a study to evaluate the ability of imaging to predict risk of cardiac death in subjects having LVEF 30-35%.

### List of Abbreviations Used in the Examples

NYHA: New York Heart Association
LVEF: left ventricular ejection fraction
IRB: Institutional Review Board
IEC: Independent Ethics Committee
SSDI: Social Security Death Index
CRF: case report form
HF: heart failure
LVAD: left-ventricular assist device
ICD: implantable cardioverter defibrillator
H/M: heart to mediastinum ratio
ROI: region of interest
CI: Confidence Interval

### EXAMPLES

### Example 1: Evaluation of [¹²³I]mIBG Imaging to Predict Risk of Cardiac Death in Subjects Having LVEF 30-35%

### Study Design & Subjects

A Phase 4, open-label, multicenter trial was carried out to investigate the prognostic usefulness of AdreView™ imaging to identify those subjects with New York Heart Association (NYHA) Class II or III HF who would die during 60 months of follow-up from the date of administration of AdreView™. HF subjects enrolled and dosed with AdreView™ in the ADMIRE-HF study (Jacobson et al. 2010 J Am Col Cardiol; 55(20): 2212-2221) were eligible to participate.

All subjects completed the informed consent form before collection of any study information. However, waiver of informed consent was requested from the Institutional Review Board (IRB)/Independent Ethics Committee (IEC) for subjects who could not be located; for those who, in the course of attempts to contact them, were determined to have died; and for those who were determined to be alive but declined to provide informed consent. For deceased subjects and those who could not be located, the IRB/IEC was requested to allow the recording of survival status and information from medical institution records and publicly available sources (death certificates, media reports, Social Security Death Index [SSDI], etc.) on the case report form (CRF). For subjects who declined to provide informed consent, the IRB/IEC was requested to allow recording on the CRF that the subject was alive.

Subject status was determined at a single point in time, specifically the date on which the subject or another individual with knowledge of the subject's survival status was contacted. As per protocol, in-person visits were required only to verify details of a life-saving intervention.

### Collection of Follow-Up Information

If the subject was determined to be deceased, information was sought from a designated medical care provider, as well as from publicly available sources such as the SSDI, depending upon the provisions of the approved informed consent form or the IRB/IEC waiver. For subjects confirmed to have died subsequent to last follow-up in the ADMIRE-HF study, the investigator endeavored to determine date and cause of death in order to categorize the death as cardiac (due to HF, sudden death, myocardial infarction, or other causes) or non-cardiac.

If the subject was alive at the date of last contact in the ADMIRE-HF study, the investigator indicated if either of the following categories of events occurred subsequent to the subject's latest follow-up:

### Category 1: Change in Cardiac Physiology

Cardiac transplant
Insertion of left-ventricular assist device (LVAD)

### Category 2: Potentially Life-Threatening Arrhythmic Event

Resuscitated cardiac arrest.
Appropriate implantable cardioverter defibrillator (ICD) discharge (anti-tachycardia pacing or defibrillation).

The efficacy population included all subjects who received an administration of AdreView™, were successfully scanned in the ADMIRE-HF study, were not withdrawn because of protocol violations, and met all entrance criteria for the present study.

The primary analysis tested the prognostic value of the numerical H/M ratio on planar AdreView™ imaging, dichotomized as either ≥1.60 or <1.60, for death in HF subjects during 60 months of follow-up. The relative hazard at time t was assessed for these subjects to assess the prognostic value of the numerical H/M ratio on planar AdreView™ imaging by a proportional hazards model as described below. A univariate Cox proportional hazards model was fitted to the time to death for each subject. The time to death was the response variable of interest and was measured in days. The Cox proportional hazards model was used to assess relative hazard for death at time t for the subjects in the 2 groups based on the H/M ratio (<1.60 and ≥1.60), denoted as low and high.

The efficacy analyses using planar scintigraphy used the derived consensus reader interpretation for the H/M ratio. This single interpretation was based upon the 3-hour, 50-minute H/M ratio accepted by at least 2 of the readers. If a different H/M ratio was accepted by each reader, then the mean value was used in the analyses. If 2 readers judged an image as non-diagnostic, the subject was considered non-diagnostic and was excluded from the analyses.

For each planar image, myocardial and mediastinum regions of interest (ROIs) were drawn and the H/M ratio calculated as per the methodology provided by the sponsor and as described below:
(1) Draw an irregular ROI defining the epicardial border of the heart. If the epicardial border cannot be defined because all or the majority of the myocardium is not visualized, draw the ROI based upon the presumed location of the heart, using the medial aspects of the left and right lower lung for anatomical guidance.
(2) Draw a horizontal line to mark the estimated location of the lung apices. If the most superior aspect of the image does not include the lung apices (because of limited field of view for a small gamma camera), draw this line at the top of the image display.
(3) Draw a vertical line approximately equidistant from the medial aspects of the right and left lung.
(4) Examine the counts for the 12 pixels along the vertical line starting 4 pixels below the intersection point with the horizontal line determined in step 2, and identify the pixel with the lowest counts. If more than one pixel has this same number of counts, choose the most superiorly located pixel.
(5) Using the pixel defined in step 4 as the center, draw a square ROI of 7×7 dimensions.
(6) Calculate the H/M ratio by dividing the counts/pixel in the total myocardium ROI determined in step 1 by the counts/pixel in the 7×7 pixel mediastinal ROI determined in step 5.

### Results

Confidence Interval (CI) was calculated using the exact Clopper-Pearson method for binomial proportion.

TABLE 1
All-cause mortality based on On-Site Left Ventricular Ejection
Fraction Categorization and the Adreview ™ Uptake
H/M Ratio at 3 Hours 50 Minutes Post Dose on Planar
Scintigraphy Categorization (<1.60 vs. >=1.60).

| | H/M (<1.60) | H/M (>=1.60) |
|---|---|---|
| Number of Subjects | 285 | 101 |
| Number of Deaths | 124 | 20 |
| Mortality Rate | 124/285 = 43.5% | 20/101 = 19.8% |
| Mortality Rate 95% CI | 37.7, 49.5 | 12.5, 28.9 |

TABLE 2
Cardiac Mortality Based on On-Site Lab Left Ventricular Ejection
Fraction Categorization and the Adreview ™ Uptake H/M
Ratio at 3 Hours 50 Minutes Post Dose on Planar Scintigraphy
Categorization (<1.60 vs. >=1.60).

| | H/M (<1.60) | H/M (>=1.60) |
|---|---|---|
| | N1 = 760 | N1 = 201 |
| Number of Subjects | 285 | 101 |
| Number of Cardiac Deaths | 47 | 5 |
| Cardiac Mortality Rate | 47/285 = 16.5% | 5/101 = 5% |
| Cardiac Mortality Rate 95% CI | 12.4, 21.3 | 1.6, 11.2 |

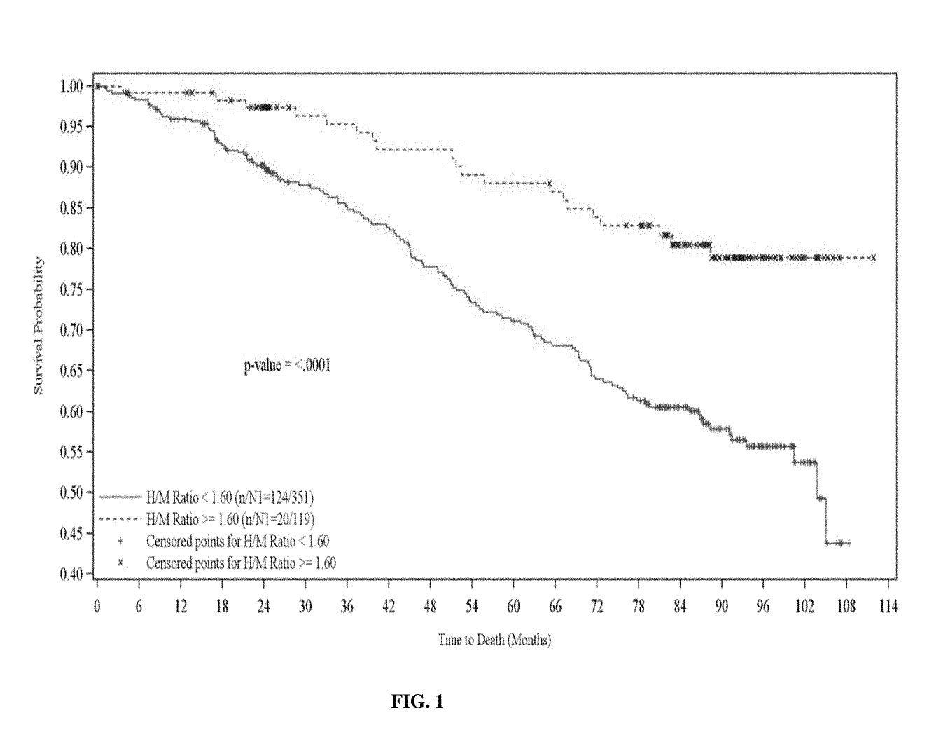

Additionally, Kaplan Meier curves for all cause and cardiac mortality at 5 years follow up and for time to first arrhythmic event showed significant differences between patients with H/M ratio <1.6 and those with ratios ≥1.6 in the LVEF strata of 30-35% by onsite readings. This is illustrated in FIGS. 1 (all-cause mortality), 2 (cardiac death) and 3 (first arrhythmic event).

1. A method comprising:
(i) obtaining an in vivo image with meta-[¹²³I]iodobenzylguanidine ([¹²³I]mIBG) of a human subject wherein said subject has a left ventricular ejection fraction (LVEF) of 30-35% and New York Heart Association (NYHA) symptoms of class II or above;
(ii) measuring [¹²³I]mIBG uptake in said subject wherein said measuring comprises defining in said in vivo image a heart region of interest (ROI) and a mediastinum ROI;
(iii) calculating the ratio of [¹²³I]mIBG uptake between said heart ROI and said mediastinum ROI (H/M ratio);
(iv) making a clinical decision based on whether or not H/M is less than 1.6.

2. The method as defined in claim 1 wherein step (i) comprises intravenous injection of a radiopharmaceutical composition comprising [¹²³I]mIBG.

3. The method as defined in claim 1 wherein step (i) comprises planar scintigraphy.

4. The method as defined in claim 1 wherein step (i) comprises obtaining early and late anterior planar scintigraphic images.

5. The method as defined in claim 4 wherein said early anterior planar scintigraphic image is obtained at 15 minutes following injection of said radiopharmaceutical composition comprising [¹²³I]mIBG.

6. The method as defined in either claim 4 wherein said late planar scintigraphic image is obtained at 3 hours and 50 minutes following injection of said radiopharmaceutical composition comprising [¹²³I]mIBG.

7. The method as defined in claim 1 wherein said heart can be visualised in said in vivo image and said heart ROI is defined by the epicardial border of the heart.

8. The method as defined in claim 1 wherein said heart cannot be visualised in said in vivo image and said heart ROI is defined by the presumed location of the heart.

9. The method of claim 1 wherein said mediastinum ROI is defined within the superior mediastinum.

10. The method as defined in claim 1 wherein a mean count density is obtained for each of said heart ROI and said mediastinum ROI.

11. The method as defined in claim 1 wherein said human subjects have NYHA symptoms of class III or above.

12. The method as defined in claim 1 wherein said clinical decision is not to implant a cardioverter-defibrillator (ICD) where said H/M ratio is greater than or equal to 1.6.

13. The method as defined in claim 1 wherein said clinical decision is selection of subjects for a clinical trial.

14. Use of [¹²³I]mIBG in a method for guiding ICD therapy in a group of patients having LVEF of 30-35% and NYHA symptoms of class II or above wherein said method is as defined in claim 1.

15. The use as defined in claim 14 wherein said patients have NYHA symptoms of class III or above.

16. A composition comprising [¹²³I]mIBG for use in the method as defined in claim 1.
